(19) United States
(12) Patent Application Publication
(10) Pub. No.: US 2014/0024725 A1
(43) Pub. Date: Jan. 23, 2014

(54) MODULATORS FOR TRPA1 FOR THE TREATMENT OF ROSACEA

(71) Applicants: Jéróme Aubert, Grasse (FR); Martin Steinhoff, San Francisco, CA (US)

(72) Inventors: Jéróme Aubert, Grasse (FR); Martin Steinhoff, San Francisco, CA (US)

(21) Appl. No.: 13/997,134

(22) PCT Filed: Dec. 19, 2011
(86) PCT No.: PCT/EP2011/073299

§ 371 Date: Oct. 4, 2013

Related U.S. Application Data
(60) Provisional application No. 61/457,076, filed on Dec. 22, 2010.

(51) Int. Cl. A61K 45/00; G01N 33/50

(52) U.S. Cl. CPC A61K 45/00; G01N 33/50
USPC 514/789; 435/7.92; 260/1

(57) ABSTRACT

An in vitro method of screening candidate compounds for the preventive or curative treatment of rosacea is described. The method can include determining the capacity of a compound to modulate the expression or activity of the Transient Receptor Potential (TRP) cation channel, subfamily A, member 1(TRPA1), as well as the use of modulators of the expression or activity of this transcription factor for the treatment of rosacea. The method can also include in vitro diagnosis or prognosis of this pathology.

### BACKGROUND

Rosacea is a common, chronic and progressive inflammatory dermatosis related to vascular relaxation. It mainly affects the central part of the face and is characterized by redness of the face or hot flushes, facial erythema, papules, pustules, telangiectasia and sometimes an eye injury, also called ocular rosacea. In serious cases, particularly in men, the soft tissue of the nose may swell and produce a bulbous swelling known as rhinophyma. Rosacea generally occurs from the ages of 25 and 70, and it is much more common in individuals with a light complexion. It affects more particularly women, although this condition is generally more serious in men (Rosacea: Diagnosis and Management, Frank C. Powell, InformaHealthcare; New-York 2009.) Rosacea is chronic and persists for years with periods of exacerbation and remission. Rosacea was originally called ‘acne rosacea’ because its papules (points of slight raising of the skin) and its inflammatory pustules (pus scabs) greatly resemble those of common acne.

The result of this facial vascular anomaly is a permanent oedema of the dermis which may accompany an increased colonization with Demodex folliculorum, a parasite usually found in the follicles of the face.

Many factors may also be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

According to the National Rosacea Society, rosacea may be classified into four subtypes plus one variant (Erythematotelangiectatic rosacea, papulopustular, phymatous, eyepiece and a variant called granulomatous rosacea).

These subtypes are listed bellow.

Subtype 1: Erythematotelangiectatic Rosacea.

Subtype 1 is characterized by flushing and persistent central facial erythema. Telangiectases are common but not essential for the diagnosis. Central facial edema, burning sensations and scales are also reported symptoms. Conventionally, the patients have erythrosis spasms due to the sudden dilation of the arterioles of the face, which then take on a congestive, red appearance. These spasms can be triggered by emotions, meals and temperature changes.

Subtype 2: Papulopustular Rosacea.

Subtype 2 is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Burning sensations may be reported by patients with papulopustular rosacea. This subtype has often been seen after or in combination with subtype 1, including the presence of telangiectases. The telangiectases may be obscured by persistent erythema, papules, or pustules. Some patients may also have red cheeks and forehead oedema.

Subtype 3: Phymatous Rosacea.

Subtype 3 includes skin thickening, irregular surface nodularities. Rhinophyma is the most common presentation, but phymatous rosacea may occur in other locations, including the chin, forehead, cheeks, and ears. Patients with this subtype may also have enlarged and prominent follicle openings. This subtype has frequently been observed after or in combination with subtypes 1 or 2, including persistent erythema, telangiectases, papules, and pustules. In the case of rhinophyma, these additional stigmata may be especially pronounced in the nasal area.

Subtype 4: Ocular Rosacea.

The diagnosis of ocular rosacea should be considered when a patients eyes have one or more of the following signs and symptoms : watery or bloodshot appearance (interpalpebral conjunctival hyperemia), foreign body sensation, burning, dryness, itching, light sensitivity, blurred vision, telangiectasia of the conjunctiva and lidmargin, or lid and periocular erythema, blepharitis, conjunctivitis, Meibomian gland dysfunction. These signs or symptoms occur before, during or after the onset of cutaneous signs. Ocular rosacea is most frequently diagnosed when cutaneous signs and symptoms of rosacea are also present. However, skin signs and symptoms are not a prerequisite to the diagnosis, and limited studies suggest that ocular signs and symptoms may occur before cutaneous manifestations in up to 20% of patients with ocular rosacea.

Granulomatous Rosacea.

There is also a granulomatous variant of rosacea, characterized by yellow, brown or red, indurated papules or nodules, and monomorphic damage in papules. Other signs of rosacea may also occur.

Of course, the pathological manifestations of rosacea vary depending on the subtype of the disease. However patients may have characteristics of different subtypes at the same time. It is also known that the disease does not necessarily progress from a subtype to another (Wilkin et al., 2002, J. AM. Acad. Dermatol. Vol. 46, pages 584-587).

Several treatments exists, but there is always a need of novel compounds as well as new diagnostic methods and new monitoring methods in order to treat more efficiently patients suffering from rosacea.

TRPs

The family of closely related cation channels, denoted Transient Receptor Potential (TRP) are known to be molecular sensors for distinct pain, temperature, chemaesthesis, and taste modalities. The first implication of TRP channels in pain and sensation was emphasized by the identification of TRPV1 at the genetic and functional level (Caterina et al; 1997).

Among the TRPs family, the transient receptor potential cation channel, subfamily A, member 1, also known as TRPA1, is a protein which in humans is encoded by the TRPA1 (and in other species by the Trpa1) gene.

TRPA1, contains 14 N-terminal ankyrin repeats and is believed to function as a mechanical stress sensor. The specific function of this protein has not yet been determined; however, studies indicate the function may involve a role in signal transduction and growth control.

The invention relates to the use of TRPA1 modulators and especially antagonists, diagnostic application and monitoring application involving TRPA1, in order to treat, diagnose and monitor rosacea in patients.

Also the invention relates to screening methods useful for determining the capability of a compound to inhibit TRPA1 activity.

Diagnostic Applications:

A first aspect of the invention is an in vitro method of diagnosis or of monitoring the development of rosacea in a subject, comprising comparison of the expression at the mRNA or protein levels of TRPA1 in a subject's biological sample, relative to a control subject.

The expression of the protein can be determined by an assay of the TRPA1, classically but non limitative ways could be an immunohistochemical test or immunoassay, for example by ELISA assay.

In order to measure the expression of the gene or the amount of corresponding mRNA, a man skilled in the art could use any known method in this field.

Within the scope of diagnosis, the ‘control’ subject is a ‘healthy’ subject.

Within the scope of monitoring the development of rosacea, the ‘control subject’ refers to the same subject at a different time, which preferably corresponds to the start of the treatment (T0).

By measuring the difference in expression or activity of TRPA1, it is notably possible to monitor the efficacy of a treatment, in particular, a treatment with a modulator of the TRPA1, as envisaged below, or by other treatments prescribed for rosacea patients.

Monitoring Applications:

Another aspect of the present invention features an in vitro method of determination of a subject's likelihood of developing rosacea, comprising comparison of the expression at the mRNA or protein levels of TRPA1 in a subjects biological sample, relative to a control subject

Once again, the expression of the protein can be determined by an assay of TRPA1, classically but non limitative ways could be an immunohistochemical test or immunoassay, for example by ELISA assay.

In order to measure the expression of the gene, or the amount of corresponding mRNA, a man skilled in the art could use any known method in this field.

The subject tested is, in this case, an asymptomatic subject, not displaying any skin disorder associated with rosacea.

The ‘control’ subject, in this method, means a ‘healthy’ subject or reference population.

Detection of this susceptibility makes it possible to start preventive treatment and/or increased monitoring for the signs associated with rosacea.

In these methods of in vitro diagnosis or prognosis, the biological sample tested can be any sample of biological fluid or a sample from a biopsy.

Preferably the sample can, however, be a preparation of skin cells, obtained for example by biopsy.

Modulators and Their Uses:

Another important aspect of the invention relates to modulators of TRPA1 to target neurogenic inflammation and their use in the treatment or prevention of rosacea

These modulators of TRPA1 may be used for the preparation of a medicinal product intended for the preventive and/or curative treatment of rosacea.

Thus, a method of preventive and/or curative treatment of rosacea is exemplified here, the said method comprising the administration of a therapeutically effective amount of a modulator of TRPA1 to a patient needing the said treatment.

Preferably, said modulators are inhibitors (or antagonists) of TRPA1 as they may hold promise in limiting the deleterious effects of rosacea symptoms.

The modulator could be formulated within pharmaceutical compositions, together with a pharmaceutically acceptable excipient.

These compositions can be administered either by the enteral, parenteral, or topical route.

Preferably, the pharmaceutical composition is applied topically.

For oral administration, the pharmaceutical composition can be in the form of tablets, capsules, sugar-coated pills, syrups, suspensions, solutions, powders, granules, emulsions, suspensions of microspheres or nanospheres or lipid or polymer vesicles providing controlled release.

For parenteral administration, the pharmaceutical composition can be in the form of solutions or suspensions for infusion or for injection.

For topical application, the pharmaceutical composition is more particularly useful for the treatment of the skin, mucosae and scalp and can be in the form of unguents, creams, milks, ointments, powders, impregnated tampons, solutions, gels, sprays, lotions or suspensions.

It can also be in the form of suspensions of microspheres or nanospheres or lipid or polymer vesicles or polymer patches or hydrogels providing controlled release.

This composition for topical application can be in anhydrous form, in aqueous form or in the form of an emulsion.

In a preferred variant, the pharmaceutical composition is in the form of a gel, a cream or a lotion.

As a non limitative example, the list in the table 1 below shows TRPA1 antagonist compounds that can be used for the treatment of rosacea, more particularly for the treatment of erythematotelangiectatic rosacea.

Examples

[symbol]

1. A medicament comprising a modulator of TRPA1 for treating rosacea.

2. The medicament as defined by claim 1, wherein the modulator comprises an antagonist of TRPA1.

3. The medicament as defined by claim 1, wherein the rosacea is selected from the group consisting of erythematotelangiectatic rosacea, papulopustular rosacea, phymatous rosacea, ocular rosacea, and granulomatous rosacea.

4. The medicament as defined by claim 1, wherein the rosacea is rythematotelangiectatic rosacea.

5. An in vitro method of diagnosing or monitoring the development of rosacea in a subject, the method comprising comparing expression or activity of TRPA1, or expression of its gene or activity of at least one of the promoters thereof, in a subject's biological sample with expression or activity levels in a biological sample of a control subject.

6. A regime or regimen for treating rosacea, the regime or regimen comprising administering to a subject in need of such treatment, for such period of time as required to elicit a desired response, a thus effective amount of a modulator of TRPA1.

7. The regime or regimen as defined by claim 6, wherein the rosacea is selected from the group consisting of erythematotelangiectatic rosacea, papulopustular rosacea, phymatous rosacea, ocular rosacea, and granulomatous rosacea.

8. The regime or regimen as defined by claim 6, wherein the rosacea is erythematotelangiectatic rosacea.
